(19) United States
(12) Patent Application Publication
(10) Pub. No.: US 2024/0315537 A1
(43) Pub. Date: Sep. 26, 2024

(54) STEREOSCOPIC VIDEO ENDOSCOPE AND VIDEO ENDOSCOPE SYSTEM

(71) Applicant: OLYMPUS Winter & Ibe GmbH, Hamburg (DE)

(72) Inventors: Sebastian Berkner, Groenwohld (DE); Andreas Mueckner, Schwarzenbek (DE); Mario BOCK, Hamburg (DE); Konrad ANDERS, Hamburg (DE)

(73) Assignee: OLYMPUS Winter & Ibe GmbH, Hamburg (DE)

(21) Appl. No.: 18/607,878

(22) Filed: Mar. 18, 2024

Related U.S. Application Data
(60) Provisional application No. 63/453,228, filed on Mar. 20, 2023.

(51) Int. Cl. A61B 1/00; A61B 1/05; A61B 90/00

(52) U.S. Cl. CPC A61B 1/00193; A61B 1/00009; A61B 1/00096; A61B 1/00186; A61B 1/051; A61B 2090/367

(57) ABSTRACT

A stereoscopic video endoscope including a main body, an elongate shaft, and a video camera disposed at a distal end of the elongate shaft. The video camera including a first optical lens system and a first imaging chip, the first optical lens system and the first imaging chip being configured to acquire a first image of a structure of interest from a first perspective; and a second optical lens system and a second imaging chip, the second optical lens system and the second imaging chip being configured to acquire a second image of the structure of interest from a second perspective. The first perspective and the second perspective being offset from each other by an angle. The first imaging chip and the second imaging chip differ from each other in at least one of spatial resolution, color resolution, or spectral sensitivity.

### CROSS-REFERENCE TO RELATED APPLICATION

The present application is based upon and claims the benefit of priority from U.S. Provisional Application No. 63/453,228 filed on Mar. 20, 2023, the entire contents of which is incorporated herein by reference.

### BACKGROUND

### Field

The present disclosure relates to video endoscopy. More specifically, the present disclosure relates to stereoscopic video endoscopes and video endoscope systems.

### Prior Art

In modern medicine, endoscopes are used for examination and treatment of internal cavities or passageways of a patient. Therefore, endoscopes usually comprise an elongated shaft which can be inserted into the cavity or passageway under examination through a natural body orifice or a surgical incision. Similar endoscopes are also used for technical inspection of machinery, where structures to be inspected are difficult to access.

Modern video endoscopes comprise a video camera disposed at a distal end of the shaft, configured to acquire video images of a structure of interest, which may be a cavity or passageway under examination, or objects located therein. A video camera of a video endoscope usually comprises an objective lens system and an imaging chip for converting an image of the structure of interest, which is projected onto a surface of the imaging chip through the objective lens system, into electronic signals like video signals.

In stereoscopic video endoscopes, the video camera is configured to acquire stereoscopic or 3D images. Therefore, the video camera usually comprises a first optical lens system and a first imaging chip, configured to acquire a first image of a structure of interest from a first perspective, and a second optical lens system and a second imaging chip, configured to acquire a second image of the structure of interest from a second perspective. The first perspective and the second perspective are usually offset from each other by an angle.

Such stereoscopic video endoscopes are, for example, known from U.S. Patent Application no. 2021/0219825.

The video images acquired by the first imaging chip and the second imaging chip are usually transmitted to a control device (controller), where they are converted into a single stereoscopic or 3D image by an image processing unit. The 3D image can then be provided for viewing on a 3D monitor or a virtual reality headset.

The stereoscopic video endoscope and the control device, also referred to as a camera control unit (camera controller), together form a video endoscope system.

Due to the two imaging chips, stereoscopic video endoscopes are expensive. It would be desirable to provide stereoscopic video endoscopes which can be manufactured with reduced costs without limiting the image quality or resolution. It would further be desirable to provide stereoscopic video endoscopes having extended imaging capability without increasing manufacturing costs.

### SUMMARY

The present disclosure provides a stereoscopic video endoscope having a main body, an elongate shaft, and a video camera disposed at a distal end of the elongate shaft. The video camera comprises a first optical lens system and first imaging chip, the first optical lens system and the first imaging chip being configured to acquire a first image of a structure of interest from a first perspective. The video camera further comprises a second optical system and a second imaging chip, the second optical lens system and second imaging chip being configured to acquire a second image of the structure of interest from a second perspective. The first perspective and the second perspective are offset from each other by an angle. The first imaging chip and the second imaging chip differ from each other in at least one of spatial resolution, color resolution, or spectral sensitivity.

The two images acquired by the first and second imaging chips of a stereoscopic video endoscope contain a significant amount of redundant data. While the two images are required from different perspectives, which is necessary to create a stereoscopic effect, the total information content of the stereoscopic image, into which the two images are converted, is much less than the sum of the information content of both individual images.

A stereoscopic image with the same information content as the stereoscopic image provided by a known stereoscopic video endoscope can be obtained by combining a first image acquired by a first imaging chip, being the same as the first imaging chip of a known stereoscopic video endoscope, with a second image acquired by a second imaging chip having a lower spatial or a lower color resolution than the first imaging chip.

In a stereoscopic video endoscope according to the present disclosure, the first image being obtained by the first imaging chip can provide structural and chromatic information with high spatial resolution and high color resolution, where the second image obtained by the second imaging chip can mainly provide disparity information between corresponding structures in the first and second images, which are used for creating the stereoscopic effect.

In some embodiments, the video camera may comprise at least one optical element which can be shared by the first optical lens system and the second optical lens system. At least one of the first optical lens system and the second optical lens system may comprise a prism for deflecting a beam path of light traveling through the respective optical lens system.

The first imaging chip may be a polychromatic imaging chip. The first imaging chip may comprise a plurality of first color filters arranged in a pattern. The pattern may be a Bayer pattern with red, green, and blue color filters.

The second imaging chip may be a monochromatic imaging chip. A pixel size of the second imaging chip may be larger than a pixel size of the first imaging chip. A pixel pitch of the second imaging chip may be larger than a pixel pitch of the first imaging chip.

In some embodiments, the second imaging chip may differ from the first imaging chip in that the second imaging chip as a higher sensitivity in an NIR wavelength range than first imaging chip. With an enhanced sensitivity in the NIR wavelength range, the second imaging chip can provide higher sensitivity in fluorescence imaging, in such embodiments, the total information content provided by the first and second images can be increased compared to known stereoscopic video endoscopes due to the reduction of redundancy.

The present disclosure further provides a video endoscope system comprising a stereoscopic video endoscope according to the above description, and an image processor comprising hardware. The image processor is configured to receive video image signals from the stereoscopic video endoscope, the video image signals representing a first video image acquired by the first imaging chip and a second video image acquired by the second imaging chip. The image processor is configured to selectively apply one of a plurality of image processing algorithms to the video image signals for combining the first video image and the second video image into a composite third video image.

The plurality of image processing algorithms may comprise a first algorithm and a second algorithm, the first algorithm configured to combine the first video image and the second video image into a 3D composite image, and the second algorithm configured to combine the first video image and the second video image into a 2D composite image of enhanced spatial resolution. The plurality of image processing algorithms may further comprise a third algorithm, configured to combine the first video image and the second video image into a 2D composite image of enhanced spectral resolution.

### BRIEF DESCRIPTION OF THE DRAWINGS

The subject of this disclosure is further described in more detail by some exemplary embodiments and drawings. Such embodiments and drawings are only provided for better understanding the concept of the disclosure, without limiting the scope of protection, which is defined by the appended claims.

It will be appreciated that the following drawings are not necessarily drawn to scale. The drawings only show those elements necessary for understanding the subject of the disclosure and may be simplified for better grasping the underlying concepts.

In the drawings:

FIG. 1 illustrates a video endoscope system:

FIG. 2 illustrates an objective lens system of a stereoscopic video endoscope;

FIG. 3 illustrates first and second imaging chips of a stereoscopic video endoscope;

FIG. 4 illustrates first and second imaging chips of a further stereoscopic video endoscope;

FIG. 5 illustrates an image processing algorithm;

FIG. 6 illustrates a further image processing algorithm; and

FIG. 7 illustrates an even further image processing algorithm.

### DETAILED DESCRIPTION

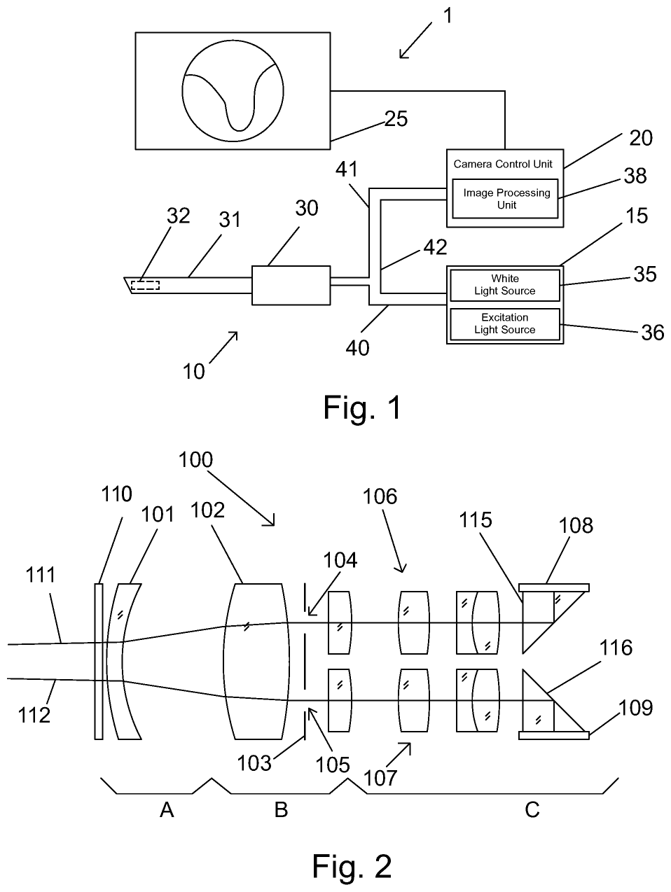

FIG. 1 shows a video endoscope system 1 with a stereoscopic video endoscope 10, a light source unit 15, a camera control unit (controller) 20, and a stereoscopic monitor 25.

The stereoscopic video endoscope comprises a main body 30, an elongate shaft 31, and a video camera 32 disposed at a distal end of the elongate shaft 31. The video camera 32 is shown in dashed lines as it is normally hidden inside the elongate shaft 31.

The light source unit 15 comprises a white light source 35 and an excitation light source 36. The white light source 35 may comprise a halogen bulb, a Xenon lamp, or one or more LED light sources. Depending on the required wavelength of excitation light, the excitation light source 36 may comprise an Hg lamp, an UV LED, an IR LED, a laser diode, or the like. Instead of separated light sources for white light and excitation light, the light source unit 15 may comprise a broadband light source and associated filters that can be selectively activated or deactivated to allow passing of white light and/or excitation light. In other embodiments not shown in the drawings, the light source unit 15 may only comprise the white light source 35.

Light from the light source unit 15 is directed to the stereoscopic video endoscope 10 through a light guide cable 40.

The camera control unit 20 provides control signals to, and receives video signals from, the video camera 32 through a signal cable 41. The camera control unit 20 comprises an image processing unit (image processor) 38, in which the video signals received from the video endoscope 10 are processed for display on the stereoscopic display 25. In addition to being displayed, the video signals may also be recorded on a memory (not shown) for later retrieval and evaluation.

Each of the camera control unit 20 and the image processing unit 38 may be separably formed or formed as a single controller and may be a CPU, computer or the like running software stored in an associated memory and/or may be dedicated circuits for performing the functions described herein.

The camera control unit 20 may further communicate with the light source unit 15 through a control cable 42. For example, the camera control unit 20 may send commands to the light source unit 15 to activate and/or deactivate the white light source 35, the excitation light source 36, or both.

The light guide cable 40, the signal cable 41, and the control cable 42 may be integrated into a single cable unit fixedly attached to the video endoscope 10, and releasably attached to the light source unit 15 and the camera control unit 20 through appropriate connectors like plugs, or the like. An additional control cable (not shown) may be provided between the camera control unit 20 and the light source unit 15, or the control cable 42 may be provided as separate cable not being part of the cable unit.

Suitable light source units 15 are available for example from the Olympus Corporation of the Americas, 3500 Corporate Parkway, PA 18034, Center Valley, USA.

FIG. 2 illustrates an objective lens system 100 of the video camera 4 of the stereoscopic video endoscope. The objective lens system 100 comprises a distal objective lens portion A, a transition portion B and a proximal objective lens portion C.

In the illustrated example, the distal objective lens portion A comprises only a single diverging lens 101. The transition portion B comprises a converging lens 102 and a stop 103 with apertures 104, 105 for a beam of a first image, which may be a left partial image and a beam of a second image, which may be a right partial image.

The transition portion B is adjoined by the proximal objective lens portion C, which comprises two lens systems 106, 107 that are disposed parallel to one another. A first imaging chip (image sensor) 108 and a second imaging chip (image sensor) 109 convert the first and second images projected by the objective lens system 100 into electronic signals for further processing. At the distal side, the objective lens system 100 is closed off by a window 110.

It can be seen that the objective lens system 100 comprises a first optical lens system 106 for forming the first image, and a second optical lens system 107 for forming the second image. In the shown embodiment, the first and second optical lens systems 106, 107 share the lenses 101 and 102. The path of the beams of the first image and of the second image is indicated by two central light rays 111, 112, which propagate along the optical axes of the objective lens system 100. Outside of the objective lens system, the light rays 11, 112 are offset by a small angle, which may be in the range of 2° to 5°.

In the shown embodiment, the first and second lens systems 106, 107 each comprise a prism 15, 116 for deflecting the beam paths of light travelling through the respective lens systems 106, 107. The prisms 115, 116 make it possible to arrange the imaging chips 108, 109 in a plane parallel to a longitudinal axis of the stereoscopic video endoscope 10, which simplifies arrangement of auxiliary circuitry not shown in the drawings. While FIG. 2 shows the prisms 115, 116 deflecting the respective beam paths within the drawing plane, the prisms 115, 116 may instead be arranged to deflect the beam paths at an angle to the drawing plane. In one example, the prism 115 may be arranged to deflect the beam path of the first lens system 106 to the back of the drawing plane, and prism 116 may be arranged to deflect the beam path of the second lens system 107 to the front of the drawing plane.

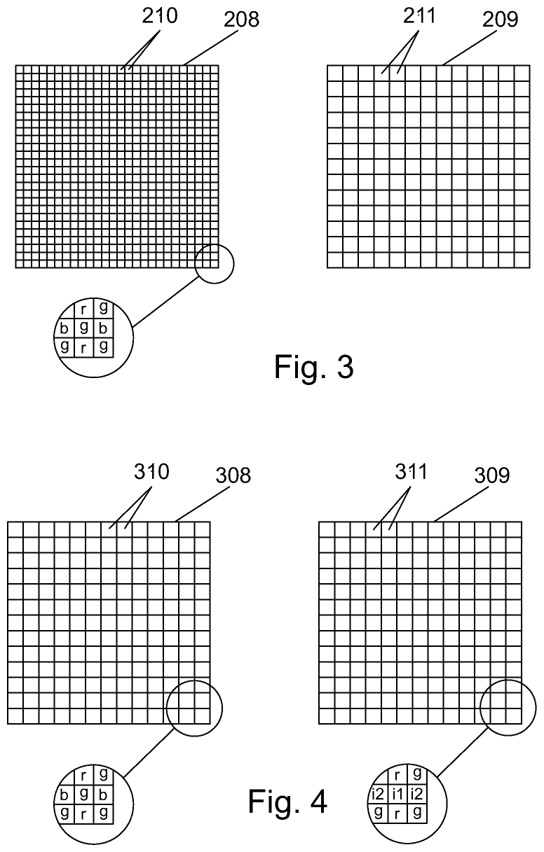

In FIG. 3, the first and second imaging chips of a possible embodiment of a stereoscopic video endoscope are shown.

In the shown embodiment, the first imaging chip 208 is a polychromatic imaging chip with a high spatial resolution. While the drawing shows a resolution of 26×26 pixels 210, the resolution will be much higher in a real stereoscopic video endoscope, e.g., 2048×1080 pixels in an HD video endoscope.

In order to provide color information, the imaging chip 208 is equipped with color filters. In the shown example, each 2×2 square of 4 pixels is covered with one red color filter “r”, two green color filters “g”, and one blue color filter “b”. Such color filter scheme is also known as “Bayer filter” By interpolation, video image signals from the first imaging chip 208 can be converted into a HD color image.

The second imaging chip 209 comprises a lower number of pixels 211, for example a quarter of the number of pixels 210 of the first imaging chip 208. Other than the first imaging chip 208, the second imaging chip 209 is not equipped with color filters. Accordingly, video signals from the second imaging chip only comprise brightness information, and therefore form a monochromatic image.

Due to the lower number of pixels, the second imaging chip 209 can have a larger pixel size and/or a larger pixel pitch than the first imaging chip 208. With a larger pixel size, the second imaging chip 209 can provide better images in low-light conditions, e.g. during fluorescence imaging. Due to the missing color filters, the second imaging chip 209 can also detect more light in the NIR wavelength range, which is often used in fluorescence imaging.

In FIG. 4, first and second imaging chips 308, 309 of a further embodiment of a stereoscopic video endoscope are shown.

Other than in the embodiment shown in FIG. 3, the first and second imaging chips 308, 309 of the present embodiment have the same number of pixels 301, 311. The second imaging chip 309 of the present embodiment is configured to provide a higher sensitivity in the NIR wavelength range than the first imaging chip 308. As an example, the second imaging chip 309 may be a Ga-based imaging chip instead of a commonly used Si-based imaging chip. Ga-based imaging chips have a higher sensitivity for NIR wavelengths.

The first imaging chip 308 is again equipped with color filters in a Bayer pattern, similar to the first imaging chip 208 of the embodiment shown in FIG. 3. The second imaging chip 309 may be equipped with color filters covering a different wavelength range. In the shown example, a 2×2 square of 4 pixels 311 of the second imaging chip 309 is equipped with one green color filter “g”, one red color filter “r”, a first IR wavelength filter “i1”, and a second IR wavelength filter “i2”. In other embodiments not shown in the drawings, the second imaging chip 309 may not be equipped with color filters but may be configured to acquire a monochromatic image.

The video signals from the first and second imaging chips are communicated to the camera control unit 20, where they are processed by the image processing unit 38. The image processing 38 unit may comprise separate computer hardware or may be implemented by software running on the computer hardware of the camera control unit. For complex video image processing operations, the image processing unit can comprise, or can be executed by, graphic processors providing a plurality of parallelly operating processor cores. In other embodiments not shown in the drawings, the image processing unit 38 may not be part of the camera control unit but may be an external unit which may be connected to the camera control unit 20 through a network. The image processing unit 38 may be provided as a cloud-based service program receiving the first and second images as an input video data stream and proving the converted image as an output video data stream.

The image processing unit 38 is configured to selectively apply one out of a plurality of image processing algorithms. The plurality of image processing algorithms may comprise a first, a second, and a third algorithm. Non-limiting examples of such algorithms are described in more detail below. The camera control unit 20 may comprise user input elements (not shown), like switches, buttons, or virtual operation elements on a touch screen, through which a used can select an algorithm to be applied.

A first algorithm is configured to use the first image and the second image for compiling a 3D composite image.

Generally speaking, a 3D image contains brightness information, color information, and depth information for a plurality of pixels. In this context, the term “depth” describes a distance of an object represented by a particular pixel from a camera with which the image has been acquired. Brightness and color information are often provided as numeric brightness values in each of three basic colors, usually red, green, and blue, and depth information may be provided as an additional numeric value. Such images are often referred to as RGBD images.

However, for presentation to a human, it is more common to provide 3D images similar to the way they are physically perceived, with a first image taken from a first perspective, and a second image taken from a second perspective, the first and second perspectives being offset by an angle. Such images are usually referred to as stereoscopic images and can readily be presented to a human by providing the first image to the left eye, and the second image to the right eye, or vice versa. For such presentation, stereoscopic display devices have been developed which use linear polarisation or synchronized shutters for selectively presenting the first and second images to a left and right eye. Such display devices usually require the use of corresponding glasses or goggles. Alternatively, stereoscopic images may be presented to a human using virtual reality headsets, wherein separate display monitors are placed before the left and right eyes of the user.

In the following, algorithms for compiling 3D images are described with relation to stereoscopic images. Those skilled in the art will readily be able to modify the described algorithms so that they are suited for compiling RGBD images instead.

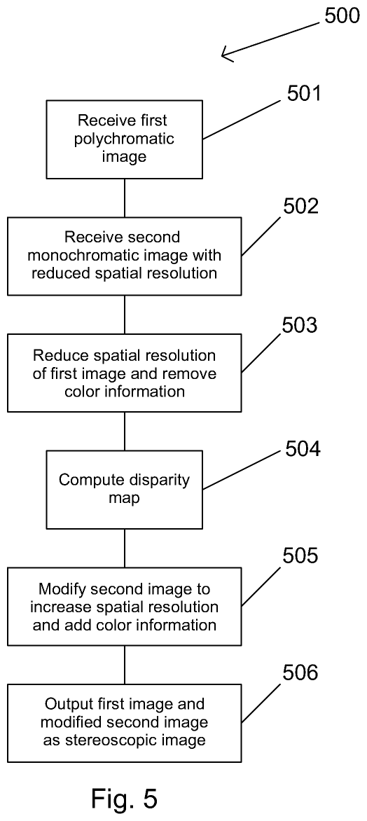

FIG. 5 shows an algorithm 500 for compiling a polychromatic stereoscopic image from a first polychromatic image with a first spatial resolution and a second monochromatic image with a second spatial resolution, the second spatial resolution being lower than the first spatial resolution.

In a first step 501, the first image is received by the image processing unit. In step 502, the second image is received by the image processing unit.

In a third step 503, the first image is modified to remove color information and to reduce the spatial resolution, so that the modified first image has the same spatial resolution as the second image. The modification can include averaging operations to combine the brightness information of separate colors and neighbouring pixels.

In a fourth step 504, a disparity map is compiled using the modified first image and the second image. The disparity map may by an array of numerical values corresponding to the pixels of the modified first image, each value indicating how far a feature represented by a particular pixel of the modified first image is shifted in the second image along a baseline direction of the stereoscopic image to be compiled. For determining respective disparity values, similarity values of small pixel groups in the modified first image and the second image may be used.

In some cases, structural features of the first image may not be visible in the second image, and vice versa, due to shading. In such cases, a signal value may be stored in the disparity map at the corresponding location.

In a fifth step 505, the disparity map is used to transfer the original spatial resolution and color information of the first image to the second image, so that a modified second image is compiled, being polychromatic and having the same spatial resolution as the first image. Herein, each pixel of the original second image may be replaced by a group of pixels from the original first image at a position shifted according to the local disparity value.

If a disparity value is not available for a particular pixel of the second image due to the corresponding structure being hidden in the first image, the respective pixels of the modified second image may be filled by interpolation. While this may result in reduced image quality in the affected areas, such reduced image quality will usually not significantly affect an endoscopic examination, as a practitioner will always navigate the endoscope to have full view of a structure of interest.

In a sixth step 506, the first image and the modified second image are output as polychromatic stereoscopic image having the same spatial resolution as the first image.

A second algorithm is configured to use the first image and the second image to compile a 2D image with enhanced spatial resolution.

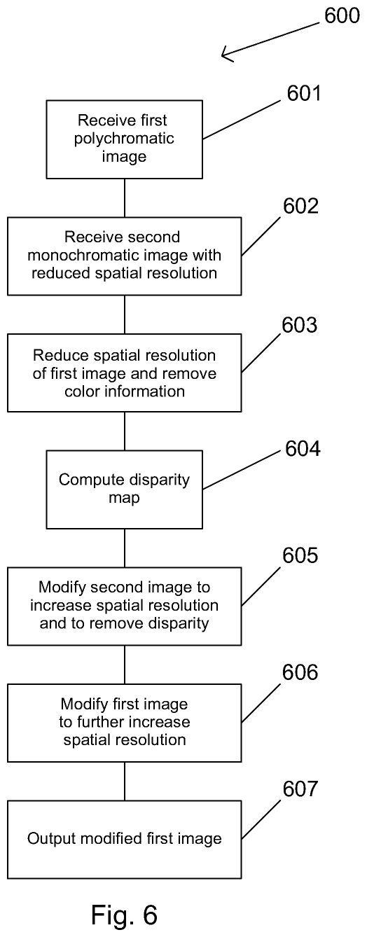

FIG. 6 shows an algorithm 600 for compiling a polychromatic 2D image with enhanced spatial resolution from a first polychromatic image having a first spatial resolution, and a monochromatic second image having a second spatial resolution, which is lower than the first spatial resolution.

In a first step 601, the first image is received by the image processing unit. In step 602, the second image is received by the image processing unit.

In a third step 603, the first image is modified to remove color information and to reduce the spatial resolution, so that the modified first image has the same spatial resolution as the second image. In a fourth step 604, a disparity map is compiled using the modified first image and the second image. The third and fourth steps 603, 604 correspond to steps 503 and 504 of the algorithm 500 and are therefore not described in detail again.

In a fifth step 605, the second image is modified to increase spatial resolution and to remove the disparity. Increase of the spatial resolution may involve interpolation, so that a smooth modified second image may be obtained. For removing disparity, each pixel of the modified second image is shifted according to the disparity value at the respective position.

Where a disparity value is not available due to shading, the respective pixels of the modified second image may be filled with a signal value so that they may be ignored in the further procedure.

In a sixth step 606, the modified second image is used to enhance the spatial resolution of the first image. First, the original first image is transformed to increase the number of pixels. In an example, each pixel of the first image is transformed into four pixels of the enhanced first image. One of the four pixels of the enhanced first image may be filled with the brightness and color information of the original pixel of the original first image. The further three pixels of the enhanced first image may be filled using interpolation of data from the surrounding pixels of the original first image and data of the original and surrounding pixels of the modified second image.

While interpolation using only surrounding pixels of the original first image would not result in real enhancement of the spatial resolution, the modified second image has been taken from a different perspective, and therefore adds true information to the enhanced first image.

In a seventh step 607, the enhanced first image is output by the image processing unit.

A third image processing algorithm may be configured to compile a composite image with enhanced spectral resolution using a first polychromatic image and a second polychromatic image.

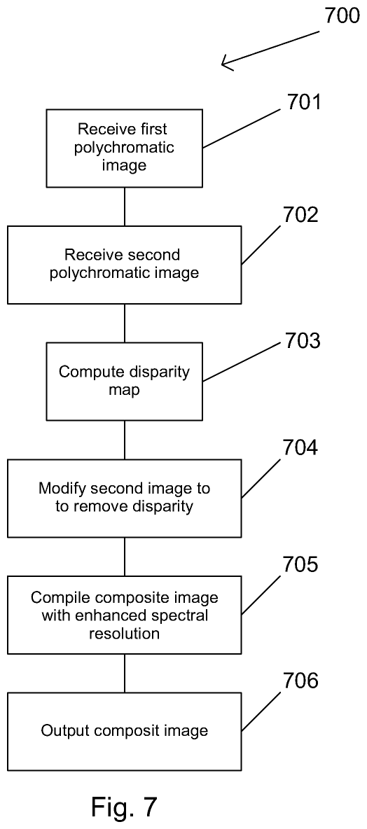

FIG. 7 shows an image processing algorithm 700 configured to compile a composite image with enhanced spectral resolution from first and second polychromatic images, wherein the first and second images cover different spectral ranges.

Again, in steps 701 and 702, the first and second images are received by the image processing unit.

As both first and second image are provided with the same spatial resolution, no further modification may be necessary before a disparity map is computed in step 703.

In a fourth step 704, the second image is modified to remove the disparity, similar to step 605 of the algorithm 600. Other than in algorithm 600, areas of the second image without available disparity values may be filled using interpolation.

In a fifth step 705, a composite image is compiled from the first image and the modified second image, using brightness and color information from each pixel of the first image and each corresponding pixel of the modified second image. For example, where the first image comprises color information using basic colors red, green, and blue, and the second image comprises color information using basic colors red, green, IR1, and IR2, the composite image may comprise color information with five basic colors red, green, blue, IR1, and IR2, wherein the blue color information is taken from the first image, the IR1 and IR2 color information is taken from the second image, and red and green color information are acquired as an average of the first and second images.

In some embodiments, the second image may be provided as monochromatic image acquired with increased sensitivity in the NIR wavelength range. In such embodiments, an IR color information may be obtained by subtracting an average brightness value of the first image, e.g. obtained by calculating a mean of the red, green and blue values, from a brightness value of the second image.

In a sixth step 706, the composite image is output by the image processing unit.

The first, second, and third algorithms described herein only serve as examples, and may easily be modified by a person skilled in the art to be fit for a particular purpose. While the algorithms have been described as deterministic algorithms, they may partially or fully employ non-deterministic methods commonly referred to as artificial intelligence (AI). In particular, image processing steps like reducing or increasing spatial resolution, calculating the disparity map, and the like, may involve use of neural networks. For the discussed image processing operations, convolutional neuronal networks (CNN) may be particularly useful.

As the image processing algorithms described herein may require significant computing power, the image processing unit 38 may not be included in the camera control unit 20 but may be provided as an external unit. In some embodiments, the image processing unit may be provided as a cloud based “on demand” service by a producer of the video endoscope system 1. Herein, the first and second images may be communicated from the camera control unit 20 to the image processing unit as an input video stream, and the image processing algorithms may be performed in a remote data center. The output images may then be transferred back to the camera control unit 20 as an output video stream. In such remote service embodiments, the total latency of the image processing, including the latency of input and output streaming, is preferably less than 100 ms, more preferably less than 50 ms.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that can fall within the scope of the appended claims.

What is claimed is:

1. A stereoscopic video endoscope comprising:
a main body,
an elongate shaft, and
a video camera disposed at a distal end of the elongate shaft, the video camera comprising:
a first optical lens system and a first imaging chip, the first optical lens system and the first imaging chip being configured to acquire a first image of a structure of interest from a first perspective; and
a second optical lens system and a second imaging chip, the second optical lens system and the second imaging chip being configured to acquire a second image of the structure of interest from a second perspective:
wherein the first perspective and the second perspective being offset from each other by an angle, and
the first imaging chip and the second imaging chip differ from each other in at least one of spatial resolution, color resolution, or spectral sensitivity.

2. The stereoscopic video endoscope of claim 1, wherein the video camera comprises at least one optical element which is shared by the first optical lens system and the second optical lens system.

3. The stereoscopic video endoscope of claim 1, wherein at least one of the first optical lens system and the second optical lens system comprises a prism for deflecting a beam path of light travelling through the respective optical lens system.

4. The stereoscopic video endoscope of claim 1, wherein the first imaging chip is a polychromatic imaging chip.

5. The stereoscopic video endoscope of claim 4, wherein the first imaging chip comprises a plurality of first color tilters arranged in a pattern.

6. The stereoscopic video endoscope of claim 1, wherein the second imaging chip is a monochromatic imaging chip.

7. The stereoscopic video endoscope of claim 1, wherein a second pixel size of the second imaging chip is larger than a first pixel size of the first imaging chip.

8. The stereoscopic video endoscope of claim 1, wherein a second pixel pitch of the second imaging chip is larger than a first pixel pitch of the first imaging chip.

9. The stereoscopic video endoscope of claim 1, wherein the second imaging chip has a higher sensitivity in an NIR wavelength range than the first imaging chip.

10. The stereoscopic video endoscope of claim 1, wherein
the first imaging chip is a polychromatic imaging chip, and
the second imaging chip is a monochromatic imaging chip.

11. The stereoscopic video endoscope of claim 1, wherein
the first imaging chip is a polychromatic imaging chip,
the first imaging chip comprises a plurality of first color filters arranged in a pattern, and
the second imaging chip is a monochromatic imaging chip.

12. A video endoscope system, comprising:
a stereoscopic video endoscope of claim 1; and
a processor comprising hardware, wherein the processor is configured to:
receive video image signals from the stereoscopic video endoscope, the video image signals representing a first video image acquired by the first imaging chip and a second video image acquired by the second imaging chip, and
selectively apply one or more image processing algorithms to the video image signals for combining the first video image and the second video image into a composite third video image.

13. The video endoscope system of claim 12, wherein the one or more image processing algorithms comprise:
a first algorithm; and
a second algorithm,
wherein the first algorithm is configured to combine the first video image and the second video image into a 3D composite image, and
the second algorithm is configured to combine the first video image and the second video image into a 2D composite image of enhanced spatial resolution.

14. The video endoscope system of claim 13, further comprising a third algorithm configured to combine the first video image and the second video image into a 2D composite image of enhanced spectral resolution.
